(19) United States
(12) Patent Application Publication
(10) Pub. No.: US 2007/0003948 A1
(43) Pub. Date: Jan. 4, 2007

(54) Semiconductor nanocrystal complexes and methods of detecting molecular interactions using same

(76) Inventors: Louise Brogan, Freeport, ME (US); Wei Lui, Guilderland, NY (US)

Correspondence Address: KENYON & KENYON LLP, 1500 K STREET N.W., SUITE 700, WASHINGTON, DC, 20005, US

(21) Appl. No.: 11/344,045

(22) Filed: Feb. 1, 2006

Related U.S. Application Data
(60) Provisional application No. 60/648,443, filed on Feb. 1, 2005.

(51) Int. Cl. C12Q 1/68; G01N 33/53; G06F 19/00; C12M 1/34

(52) U.S. Cl. 435/6; 435/7.1; 435/287.2; 702/19; 977/924

(57) ABSTRACT

A water-stable semiconductor nanocrystal complex adapted to act as a FRET donor. The present invention also provides a method of detecting molecular interactions in an aqueous solution between a FRET acceptor and a semiconductor nanocrystal complex that is a FRET donor.

### CROSS-REFERENCE TO RELATED APPLICATIONS

The present application claims priority to U.S. Provisional Application No. 60/648,443, filed Feb. 1, 2005, which is incorporated by reference herein.

### FIELD OF THE INVENTION

The present invention relates generally to water stable semiconductor nanocrystal complexes that are adapted to act as fluorescence resonance energy transfer (FRET) donors. In addition, the invention relates to methods of detecting molecular interactions using water stable semiconductor nanocrystal complexes adapted to act as FRET donors.

### BACKGROUND OF THE INVENTION

FRET occurs when two molecules of interest, each labeled with two different fluorescent dyes, are in close proximity to each other. A FRET ‘pair’ consists of a “donor” label and an “acceptor” label. In a FRET pair, the emission spectra of the donor overlaps with the absorption spectra of the acceptor. Fluorescence emission from the acceptor results if the two molecules of interest, and therefore the two labels, are in close proximity. In contrast, if the ‘pair’ is not close enough, the result is only emission from the donor. The ratio of the two emissions gives the scientist information regarding the molecular interaction.

Organic fluorophores are often used to detect molecular interactions in fluorescence-based bioassays. However, one of the major shortcomings of using organic fluorophores as donor and acceptor molecules is the FRET signal contamination that results from the spectral overlap between the donor and acceptor emission spectra (donor spectral bleedthrough—SBT) and the donor excitation of the acceptor. As such, there is a need in the art for a FRET donor that reduces SBT contamination.

### SUMMARY OF THE INVENTION

In an embodiment, the present invention provides a semiconductor nanocrystal complex adapted to act as a FRET donor. The semiconductor nanocrystal complex comprises a semiconductor nanocrystal (also referred to herein as a “quantum dot”) and a surfactant layer surrounding the semiconductor nanocrystal. The surfactant layer has a moiety with an affinity for the semiconductor nanocrystal and a moiety with an affinity for a hydrophobic solvent. The semiconductor nanocrystal complex further comprises an additional layer having a hydrophobic end for interacting with the surfactant layer and a hydrophilic end.

In an embodiment, the present invention provides a method of detecting the presence of a FRET acceptor molecule in an aqueous solution. The method comprises introducing a semiconductor nanocrystal complex that is adapted to act as a FRET donor into an aqueous solution and exciting the semiconductor nanocrystal complex. The method further comprises determining the light emission from the aqueous solution containing the semiconductor nanocrystal complex. The method further comprises detecting the presence of the FRET acceptor molecule based on the light emission.

### BRIEF DESCRIPTION OF THE DRAWINGS

FIG. 1 is a schematic illustration of an embodiment of a semiconductor nanocrystal complex of the present invention.

FIG. 2 is an exemplary conjugation method to conjugate tertiary molecules to semiconductor nanocrystal complexes of the present invention.

FIG. 3 is a schematic illustration of the fluorescence resonance energy transfer mechanism using semiconductor nanocrystals of the present invention.

FIG. 4 depicts the steps of a method of detecting molecular interactions according to the present invention.

FIG. 5A is a schematic illustration of a semiconductor nanocrystal complex (emission 566 nm) bound to A1568-s.

FIG. 5B is a normalized excitation and emission spectra of the semiconductor nanocrystal complex and A1568-s of FIG. 5A.

FIG. 6A is a microscopy image of a cell labeled with a semiconductor nanocrystal complex upon 543 nm excitation using a Zeiss 510META confocal microscope.

FIG. 6B is a microscopy image of a cell labeled with a semiconductor nanocrystal complex upon 458 nm excitation using a Zeiss 510META confocal microscope.

FIG. 6C is a microscopy image of a cell labeled with a semiconductor nanocrystal complex upon 543 nm excitation using a Zeiss 510META confocal microscope.

FIG. 6D is a microscopy image of a cell labeled with a Alexa568-streptavidin complex upon 543 nm excitation using a Zeiss 510META confocal microscope.

FIG. 6E is a microscopy image of a cell labeled with an Alexa568-streptavidin complex upon 458 nm excitation using a Zeiss 510META confocal microscope.

FIG. 6F is a microscopy image of a cell labeled with an Alexa568-streptavidin complex upon 543 nm excitation using a Zeiss 510META confocal microscope.

FIG. 6G is a microscopy image of a cell labeled with a semiconductor nanocrystal complex and a Alexa568-strptavidin complex upon 543 nm excitation using a Zeiss 510META confocal microscope.

FIG. 6H is a microscopy image of a cell labeled with a semiconductor nanocrystal complex and a Alexa568-strptavidin complex upon 458 nm excitation using a Zeiss 510META confocal microscope.

FIG. 61 is a microscopy image of a cell labeled with a semiconductor nanocrystal complex and a Alexa568-strptavidin complex upon 543 nm excitation using a Zeiss 510META confocal microscope.

FIG. 7A represent a confocal microscopy image that shows the extent of spectal bleed through correction for labeled cells using semiconductor nanocrystal complexes of the present invention.

FIG. 7B represents the total spectral bleed through due to donor excitation of the acceptor fluorophore (lane A) and donor emission bleedthrough into the acceptor channel (lane D).

### DETAILED DESCRIPTION OF THE INVENTION

The present invention relates to semiconductor nanocrystal complexes that are water stable and adapted to act as FRET donors. The semiconductor nanocrystal complexes of the present invention can have broad absorption ranges and long fluorescent light emission capabilities throughout the visible and infrared wavelength ranges. The broad absorption properties represent the fundamental nature of solid state semiconductor material. The versatility of the semiconductor nanocrystal fluorescence stems from the small, tunable quantum dot core sizes (often from 1-10 nm). At these very small sizes, semiconductor materials display unique quantum mechanic properties. For example, when light acts on a semiconductor nanocrystal, it encounters discretized energy bands specific to the semiconductor nanocrystal quantum dot core. The discretized nature of quantum dot energy bands means that the energy separation between the valence and conduction bands (the band-gap) can be altered through the variation of the size of the semiconductor nanocrystals. Predetermining the size of the nanocrystal fixes the emitted photon wavelength to a customized color, even if it is not naturally occurring—an ability limited only to nanocrystals. The small size of the semiconductor nanocrystals also contributes to the characteristic Guassian emission peaks.

The nature of the discretized energy bands encountered when light impinges on a semiconductor nanocrystal is defined by the energy separation between the valence and conduction bands, known as the bandgap, and can be altered with the addition or the subtraction of a single atom—making for a size-dependent bandgap. The size of the dot core, therefore, fixes the emitted photon at a pre-determined wavelength. The ability to control emission wavelengths in this manner is one of the most attractive properties of quantum dots and provides a design freedom not available when using organic, small molecule fluorophores.

Semiconductor nanocrystals also have unique semiconductor properties that range between those of a single molecule and those associated with bulk semiconductor materials. Following a regime known as quantum confinement, quantum dot fluorescence can be observed at a size-determined wavelength; the density of electron states of a particular quantum dot is quantized relative to its size, such that larger nanocrystals approach bulk-like semiconductor properties, and smaller nanocrystals approach those of a single molecule. The ability to control the electron states of quantum dots, and consequently controlling their fluorescence properties, gives tremendous flexibility to a biologist in designing the appropriate materials to fit a given application.

In addition to the core related optical properties, the semiconductor nanocrystal complexes of the present invention have geometrical dimensions and surface functionality that enable FRET-based detection of specific molecular interactions and water stability. The semiconductor nanocrystalline cores may be coated with a shell of a second semiconductor material. Additionally, the complexes contain a layer of water stabilizing material that is thin enough to keep the composite within the theoretical size constraints for efficient FRET to occur. Further, the water-stabilized semiconductor nanocrystals have surface chemistries that add to their usefulness in molecular detection. The semiconductor nanocrystal complexes of the present invention can be conjugated to tertiary molecules such as targeting ligands (e.g., proteins, oligonucleotides and other biomolecules) using binding chemistries. Ligand-bound semiconductor nanocrystal complexes of the present invention therefore, can be used to probe specific molecular interactions (e.g., biomolecular interactions in live cells) and can be applied to a variety of fluorescence-based detection assays, including FRET.

The semiconductor nanocrystal complexes of the present invention, when used as FRET donors paired to organic fluorophore acceptors, can be used to overcome the limitations of organic dye fluorophore FRET donor/acceptor pairs. One of the major shortcomings of using organic fluorophores as donor and acceptor molecules is the FRET signal contamination that results from the spectral overlap between the donor and acceptor emission spectra (donor spectral bleedthrough—SBT) and the donor excitation of the acceptor. The use of semiconductor nanocrystal complexes of the present invention as FRET donors reduces the SBT contamination problem directly since they show a broad donor excitation spectra that allows donor excitation while minimizing simultaneous acceptor excitation (reduced acceptor SBT), and a narrow acceptor emission, which allows the use of specific bandwith emission filters to significantly reduce the donor emission bleedthrough into the acceptor channel upon donor excitation (reduced donor SBT).

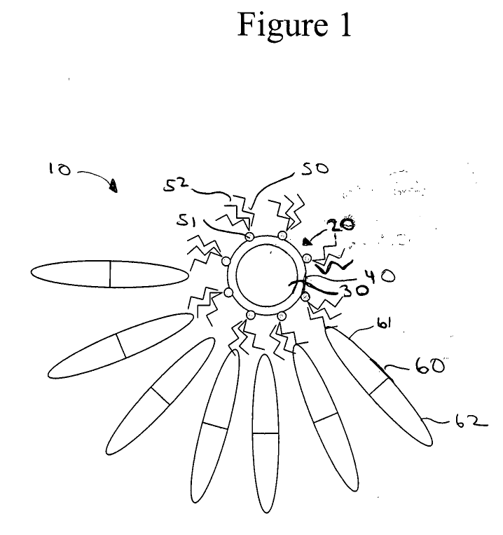

Referring to FIG. 1, in an embodiment, a water-stable semiconductor nanocrystal complex 10 comprises a semiconductor nanocrystal (also referred to herein as a “quantum dot”) 20, and a surfactant layer 50 surrounding the semiconductor nanocrystal 20, Surfactant layer 50 has a moiety 51 with an affinity for the semiconductor nanocrystal 20 and a moiety 52 with an affinity for a hydrophobic solvent. The semiconductor nanocrystal complex 10 further comprises an additional layer 60 having a hydrophobic end 61 for interacting with surfactant layer 50 and a hydrophilic end 62. Each layer of the quantum dot contributes to the fluorescence efficacy and/or particle stability of the semiconductor nanocrystal complex.

Semiconductor nanocrystal 20 comprises a semiconductor nanocrystal core 30 and an optional semiconductor nanocrystal shell 40. Semiconductor nanocrystal core 30 is typically a spherical nanoscale crystalline material (although oblate and oblique spheroids can be grown as well as rods and other shapes) having a diameter between 1 nm and 20 nm and typically but not exclusively comprising II-VI, III-V, and IV-VI binary semiconductors or ternary semiconductors. Shell 40 is preferably between 0.1 nm and 10 nm thick and preferably comprises a semiconductor material that has a lattice constant that matches or nearly matches the core and has a wider bulk bandgap than that of the core semiconductor. Examples of semiconductor materials that comprise the core and/or shell of a semiconductor nanocrystal complex of the present invention include ZnS, ZnSe, ZnTe, CdS, CdSe, CdTe, HgS, HgSe, HgTe (II-VI materials), PbS, PbSe, PbTe (IV-VI materials), AlN, AlP, AlAs, AlSb, GaN, GaP, GaAs, GaSb, InN, InP, InAs, InSb (III-V materials). Instead of or in addition to a shell, a semiconductor nanocrystal core of the present invention may have various metal layers grown around the core semiconductor nanocrystal prior to the addition of a shell around the core.

Referring back to FIG. 1, a semiconductor nanocrystal complex of the present invention further comprises a surfactant layer 50. Surfactant layer 50 comprises surfactants that are preferably organic molecules that have a moiety 51 with an affinity for the surface of the nanocrystals and another moiety 52 with an affinity for a hydrophobic solvent. Moieties are molecules or functional groups on a molecule that have a particular affinity for another molecule or another functional group on another molecule. Moieties that have an affinity to the nanocrystal surface include thiols, amines, phosphines, and phosphine oxides. Lyophilic molecules, such as TOPO (trioctyl phosphine oxide), TOP (trioctyl phosphine), and TBP (tributyl phosphine) are typically used in the synthesis of nanocrystals and can remain on the surface after preparation of the semiconductor nanocrystals or may be added or replaced by other surfactants after synthesis. The surfactants tend to assemble into a coating around the nanocrystal and enable it to suspend in a hydrophobic solvent.

Referring still to FIG. 1, a semiconductor nanocrystal complex further comprises an additional layer 60. Additional layer 60 preferably comprises lipids or polymer based small molecules including amino acids with a hydrophilic end section and a hydrophobic end section. The hydrophobic end section of the additional layer of the present invention has an affinity for the surfactant present on the surface of the semiconductor nanocrystal. The performance of semiconductor nanocrystal complexes of the present invention as dependable biological research tools is related to their ability to withstand the stringent conditions found in most cellular contexts. Oxidative stress, changes in salt concentration, pH, and temperature, as well as proteolytic susceptibility are some examples of the conditions these nanocrystals need to withstand in order to be useful in aqueous biological assays. Stabilizing the surface for water soluble applications can be achieved by layering a coat of hydrophilic material onto the semiconductor nanocrystal. The composition of this material can vary and can be selected based on the application to be used.

In addition to providing a layer of stability to the semiconductor nanocrystal, the additional layer can also provide surface exposed functional groups to facilitate the conjugation of ligands or tertiary molecules for target specific applications. This is the layer of the semiconductor nanocrystal composition that opens opportunity for the biologist to exploit an entire host of molecular interactions. For example, the semiconductor nanocrystal surfaces can be ‘tagged’ with a bio-recognition molecule (e.g., antibody, peptide, small molecule drug or nucleic acid) designed to target only the molecular signature of interest (e.g., cell surface receptor proteins, viral DNA sequences, disease antigens.) The interaction of the ‘tagged’ semiconductor nanocrystal with its target could then be visualized with the appropriate fluorescence detection and imaging equipment.

Functional groups exposed on the surface of the semiconductor nanocrystal complexes can be coupled to nucleic acids, proteins, antibodies, or small molecules and can serve as the basis for many types of in vitro detection assays. Some examples of applicable assays include, DNA/RNA assays and microarrays; high throughput screens; whole blood and tissue screening in medical diagnostics; immunoassays, dot blots and other membrane-based detection technologies. Functional groups include but are not limited to alcohol (OH), carboxylate (COOH), and amine (NH2), hydroxy, carboxyl, sulfonates, phosphates, nitrates, and any combinations thereof. Although, the additional layer is described as a single layer it is appreciated that more that one type of functional group may be present on the surface of the additional layer. In addition to comprising functional groups on its surface, the semiconductor nanocrystal complex may comprise one or more tertiary molecules.

The term tertiary molecule refers to any molecule that can be covalently coupled to the semiconductor nanocrystal complex. The coupling of tertiary molecules to the semiconductor nanocrystal complex is achieved by reacting functional groups present on the tertiary molecule with hydrophilic functional groups present on the additional layer of the semiconductor nanocrystal complex. Tertiary molecules include members of specific binding pairs such as, for example, an antibody, antigen, hapten, antihapten, biotin, avidin, streptavidin, IgG, protein A, protein G, drug receptor, drug, toxin receptor, toxin, carbohydrate, lectin, peptide receptor, peptide, protein receptor, protein, carbohydrate receptor, carbohydrate, polynucleotide binding protein, polynucleotide, DNA, RNA, aDNA, aRNA, enzyme, and substrate. Other non-limiting examples of tertiary molecules include a polypeptide, glycopeptide, peptide nucleic acid, oligonucleotide, aptamer, cellular receptor molecule, enzyme cofactor, oligosaccharide, a liposaccharide, a glycolipid, a polymer, a metallic surface, a metallic particle, and a organic dye molecule. Depending on the material used for the additional layer, the tertiary molecule may be part of the additional layer. For example, in the event that the additional layer comprises a biotin terminated lipid, then the tertiary molecule (the biotin) would be a part of the additional layer.

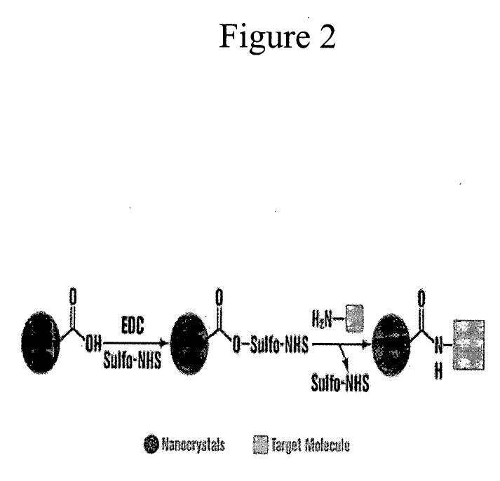

An example conjugation method to conjugate tertiary molecules to the functional groups of the additional layer of a semiconductor nanocrystal is illustrated in FIG. 2. Using this protocol, many semiconductor nanocrystals complexes that possess functional moieties suitable for conjugation to biomolecules can be prepared. The functional groups to be employed include, but are not limited to, carboxylic acids, amines, sulfhydryls and maleimides. Established protein or nucleic acid conjugation protocols can then be used to generate customized ligand-bound semiconductor nanocrystals in a straightforward manner. The method depicted in FIG. 2, uses EDC (1-ethyl-3(3-dimethylaminopropyl) carbodimide HCl) and sulfo-NHS(N-hydrocylsulfo-succinimide) to form active esters on the surface of the semiconductor nanocrystal complex. Once the unreacted EDC and sulfo-NHS are removed, a protein of interest may be added and efficiently conjugated to the semiconductor nanocrystal complex.

Most of the functional groups described are highly polar and ionizable and are likely to contribute to non-specific cell surface binding when available in high concentrations on the semiconductor nanocrystal surface. To alleviate specificity problems, the stoichiometric ratios of the molecular species composing the organic outer layer may be varied. Using an estimated approach, the number of groups added to the semiconductor nanocrystal surface can be controlled and fully saturated upon ligand conjugation. The procedure method described above enables one to attain a population of products within an acceptable range of surface-exposed functional groups.

The above described semiconductor nanocrystal complexes are adapted to act as FRET donors for the detection of molecular interactions. The ability of the semiconductor nanocrystal complexes to bind such that the core semiconductor nanocrystal is close to target molecules allows them to act as FRET donors. As such, the the distance between the semiconductor nanocrystal core or shell and the outermost surface of the semiconductor nanocrystal complex is less than 100 Å. For example, the distance between the semiconductor shell/core and the end of the tertiary molecules is less than 100 Å. Preferably, the total distance of the semiconductor nanocrystal core or shell to the outermost surface of the semiconductor nanocrystal complex is less than 90 Å, more preferably less than 80 Å, and most preferably less than 70 Å.

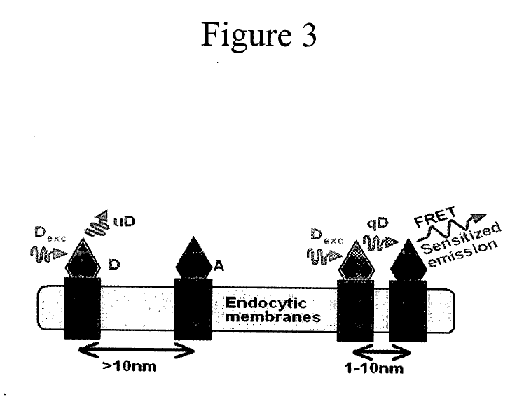

FRET determines if a donor and acceptor are within a specific distance of each other. FRET is the radiationless transfer of energy from a donor fluorophore to an acceptor fluorophore in close proximity through dipole-dipole coupling. For FRET to occur, the donor and acceptor fluorophores should have a sufficient spectral overlap, a favorable dipole-dipole orientation, a proximity of 1-10 nm and a large enough quantum yield. As can be seen in FIG. 3, FRET determines if donor (D)- and acceptor (A)-labeled molecules are within a certain distance from each other, typically 10 nm. qD stands for quenched D and indicates that the donor (D) is quenched in that example. uD or unquenched D indicates that the donor D is not quenched in that example. The excitation is depicted by exc, and emission is indicated by em. Upon energy transfer, the following events often occur: (a) donor fluorescence is quenched and acceptor fluorescence is increased (sensitized); (b) donor photobleaching rate is decreased; (c) donor excitation lifetime decreases; (d) upon acceptor photobleaching, donor fluorescence is increased (unquenching).

Semiconductor nanocrystal complexes, according to the present invention, are electronically and chemically stable with a high luminescent quantum yield. Chemical stability refers to the ability of a semiconductor nanocrystal complex to have minimal loss of fluorescence over time in aqueous and ambient conditions. Electronic stability refers to whether the addition of electron or hole withdrawing ligands substantially quench the fluorescence. Preferably, a semiconductor nanocrystal complexes would also be colloidally stable in that when suspended in organic or aqueous media (depending on the ligands) they remain soluble over time. Often times semiconductor nanocrystal complexes are not colloidally stable for more than a number or hours of days. The semiconductor nanocrystals of the present invention are colloidally stable for more than a few hours, preferably more than 2 or 3 weeks and most preferably more than 6 months. A high luminescent quantum yield refers to a quantum yield of over 10%. Preferably, the quantum yield of the semiconductor nanocrystal complex is over 20%, more preferably over 35%, and even more preferably over 50%, as measured under ambient conditions. The semiconductor nanocrystal complexes of the present invention experience little loss of fluorescence over time and can be manipulated to be soluble in organic and inorganic solvents as traditional semiconductor nanocrystals.

Additionally, semiconductor nanocrystal complexes of the present invention are prepared such that they have a high amount of energy that they allow for the estimation of efficiency energy transfer (E %). E % is based on the energy that is transferred from the donor to the acceptor, expressed as a percentage of total unquenched donor fluorescence. Using R₀, the distance at which E % is 50% (Forster distance or R₀)—determined by spectrofluorometry—E % can be used as a ‘spectroscopic ruler’, measuring the average distance between two fluorophores. Thus, for a known donor-acceptor pair, E % provides a measure of spatial proximity since it decreases rapidly with increasing distance between the two fluorophores. Preferably, semiconductor nanocrystal complexes of the present invention allow for an E % of greater than 30%, more preferably an E % greater than 40%, and most preferably a E % of greater than 50%.

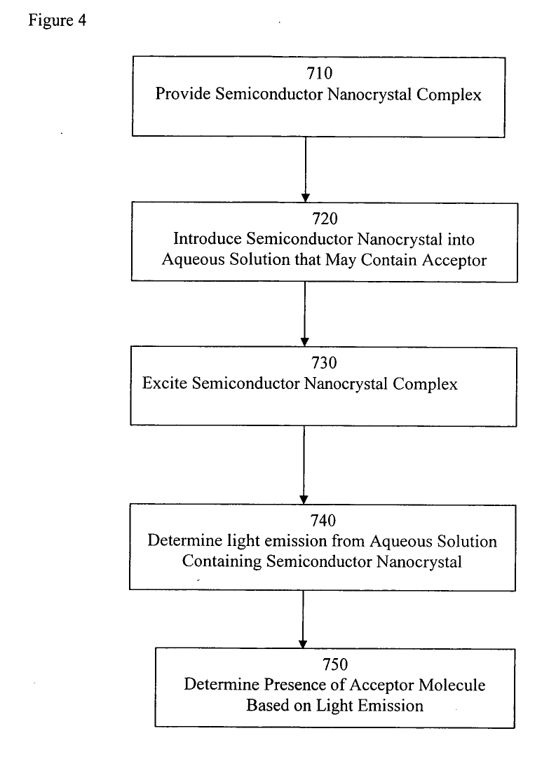

Referring to FIG. 4, the present invention also provides a method of detecting molecular interactions using a semiconductor nanocrystal of the present invention. For example, as described above, the surface of a semiconductor nanocrystal complex can be ‘tagged’ with a bio-recognition molecule (e.g., antibody, peptide, small molecule drug or nucleic acid) designed to target the molecular signature of interest (e.g., cell surface receptor proteins, viral DNA sequences, disease antigens.) The interaction of the ‘tagged’ quantum dot with its target could then be visualized with the appropriate fluorescence detection and imaging equipment.

In step 710, a semiconductor nanocrystal is prepared or provided as described above. In general, the semiconductor nanocrystal complexes is prepared with such functional groups such that when introduced into an environment containing FRET acceptor molecules, the semiconductor nanocrystal complexes will be able to act a FRET donor. Additionally, in order to act as a FRET donor, the semiconductor nanocrystal complexes are prepared such that the distance between the semiconductor nanocrystal core of the nanocrystal complexes and the potential FRET acceptor molecule will be less than 100 Å.

In step 720, the semiconductor nanocrystal complexes prepared or provided in step 710 are introduced into an aqueous solution in order to determine the presence of a molecular interaction between the semiconductor nanocrystal complex and a potential FRET acceptor molecule. The semiconductor nanocrystal complex may be selected such that upon the desired bonding between the semiconductor nanocrystal complex and the FRET acceptor molecule, the distance between the semiconductor nanocrystal core and the FRET acceptor molecule is less than 100 Å. Additionally, the donor molecule and the acceptor molecule may be selected such that the emission wavelength of the donor molecule is absorbed, at least in part, by the selected acceptor molecule. It is appreciated that if all other factors are equal, it is desirable to have as great a compatibility between the absorption spectra of the acceptor molecule and the emission spectra of the donor molecule as possible.

In step 730, the aqueous solution containing the semiconductor nanocrystal complex is illuminated with light of a wavelength that causes the semiconductor nanocrystal complex to emit light at its selected wavelength. In contrast to bulk semiconductors which display a rather uniform absorption spectrum, the absorption spectrum for semiconductor nanocrystals appears as a series of overlapping peaks that get larger at shorter wavelengths. Typically, semiconductor nanocrystals will not absorb light that has a wavelength longer than that of the first exciton peak, also referred to as the absorption onset. Thus, when using light to excite the environment comprising the semiconductor nanocrystal complexes it is desirable to use light with a wavelength less than that of the semiconductor nanocrystal complexes first exciton peak. Like all other optical and electronic properties, the wavelength of the first exciton peak (and all subsequent peaks) is a function of the composition and size of the dot. Smaller dots result in a first exciton peak at shorter wavelengths.

In step 740, the light emission from the aqueous solution is determined. One method of determining the emission from the aqueous solution is through the use of a confocal microscope. In addition to confocal microscopy, other methods to detect the emission from the aqueous solution include spectrofluorimetry. Through the use of various FRET correction algorithms, spectral bleed through from the donor or acceptor into acceptor or donor channels respectively may be corrected.

In step 750, the presence of an acceptor molecule in the aqueous solution is determined. The presence of the acceptor molecule may be determined from the emission spectrum. In the simplest situation, this may be done through the visual inspection of the aqueous solution comprising the donor molecule and possible acceptor molecules. Additionally, it may require one to use various algorithms to determine the actual energy transfer levels. Although, the above procedure is described with respect to detecting the presence of molecules, it is appreciate that the same procedure may be used to detect the concentration of molecules.

### EXAMPLE 1

### Forster Distance (R₀) for Semiconductor Nanocrystals Complexes

To address the relationship between the efficiency of energy transfer and the distance between the donor and acceptor fluorophores, the R₀ value, which represents the distance at which several donor semiconductor nanocrystal complexes to an acceptor Alexa 568 is 50% efficient, were calculated. Specifically, R₀ values for several semiconductor nanocrystal complexes as donor and Alexa568 as acceptor were calculated, assuming the donor quantum yield as 0.40-0.45 and the orientation factor k2 as ⅔ (Table 1). These R₀ values are similar to those obtained for Alexa 488-Alexa 555 pairs (70 angstroms), indicating that these semiconductor nanocrystal complexes can be highly efficient donor probes.

TABLE 1
Forster Distance (R₀) for Semiconductor
Nanocrystals Complexes - Alexa568 pairs

| Semiconductor Nanocrystal Complex | Acceptor (Alexa568) |
|---|---|
| (Material System/Emission Wavelength) | (R₀ values) |
| CdSe/ZnS (490 nm) | 53.6 Å |
| CdSe/ZnS (520 nm) | 66.3 Å |
| CdSe/ZnS (540 nm) | 75.1 Å |
| CdSe/ZnS (560 nm) | 79.8 Å |
| CdSe/ZnS (600 nm) | 76.4 Å |

R₀ values represent the distance at which FRET from the donor semiconductor nanocrystal complexes to the acceptor Alexa568 is 50% efficient.

### EXAMPLE 2

### Method of Making Semiconductor Nanocrystal Complexes

The below described procedures may be used for the development of various semiconductor nanocrystal complexes that are adapted to act as a FRET donor. Although specific amounts and temperatures are given in the below described procedures, it is appreciated that these amounts may be varied. The starting material for the below described procedure is a semiconductor nanocrystal. There are many well known ways to prepare semiconductor nanocrystals. The semiconductor nanocrystal complexes of the present invention may be prepared using any known method of semiconductor nanocrystal preparation. Additionally, semiconductor nanocrystals may be purchased from Evident Technologies. The semiconductor nanocrystals purchased from Evident Technologies work well for the purposes of the described invention. For the purpose of the procedure described below, the semiconductor nanocrystals prepared and/or purchased may be dissolved in toluene.

The below described procedure can be used to produce a semiconductor nanocrystal complex comprising a 520 nm emitting semiconductor nanocrystal with approximately 3 functional groups. Depending on the size of the semiconductor nanocrystal, the ratio of lipids described below may be varied to get the appropriate number of functional groups.

### EXAMPLE 2a

### Biotin Terminated Semiconductor Nanocrystal Complexes

Solution 1. 3 mg (1 mg/ml) CdSe/ZnS core-shell nanocrystals in a toluene solution (emission at ˜560 nm) were loaded into a 15 ml centrifuge tube, and 10 ml methanol was added. The solution was mixed, and centrifuged at 400 rpm for 3 minutes. The supernatant was removed and 3 ml of hexane was added to re-dissolve the pellet of the nanocrystals. Then 9 ml methanol was added into the tube to precipitate down the nanocrystals again. The hexane and methanol purification step were repeated one more time. The precipitate pellet was dried under air, and then re-dissolved into 1 ml chloroform.

Solution 2. 10 mg DPPE-PEG(2000) biotin lipids(1,2-Distearoyl-sn-Glycero-3-Phosphoethanolamine-N-[Biotinyl(Polyethylene Glycol)2000(Ammonium Salt)) and 60 mg mPEG2000PE ([1,2-diacyl-sn-glycero-3-phosphoethanolamine-n-methoxy(polyethylene glycol)-2000] (Ammonium Salt)) lipids were dissolved in 3 ml chloroform solution. It has been found that a ⅙ ratio of biotin lipid to ammonium salt lipid allows for an optimal number of functional groups, approximately three in this example. However, these ratios may be varied depending on the number of functional groups desired on the semiconductor nanocrystal complex.

Solution 1 and solution 2 were mixed together in a 20 ml vial and the resultant solution was dried under N₂. The vial was rotated slowly to make a thin film on the wall while the solution was drying. The vial was heated at 75° C. in a water bath for 2 minutes. To the heated vial, 5 ml deionized water which has been preheated to 75° C. was added. Then the vial was capped, and the solution was vortexed until all the nanocrystals were dissolved. Then the solution was sonicated for 1 minute.

The solution was transferred into a 15 ml centrifuge tube, centrifuged at 4000 rpm for 5 minutes. The clear supernatant was loaded into a 10 ml syringe, and was filtrated through a 0.2 μm filter. Afterwards, the filtrated solution was loaded into two 11 ml ultracentrifuge tubes, centrifuged at 65,000 rpm for 1 hour. The supernatant was removed carefully, and the precipitates re-dissolved into deionized water. The ultracentrifuge purification step was repeated one more time. Th pellets were reconstitute into 4 ml deionized water and stored at 4° C.

### EXAMPLE 2b

### Carboxy Terminated Semiconductor Nanocrystal Complexes

Solution 1. 3 mg (1 mg/ml) CdSe/ZnS core-shell nanocrystals toluene solution (emission at ˜600 nm) were loaded into a 15 ml centrifuge tube, and 10 ml methanol was added. The solution was mixed, and centrifuged at 400 rpm for 3 minutes. The supernatant was removed carefully and 3 ml of hexane was added to re-dissolve the pellet of the nanocrystals. Then 9 ml methanol was added in the tube to precipitate down the nanocrystals again. The hexane and methanol purification steps were repeated one more time. The precipitate pellet was dried, and then re-dissolved into 1 ml chloroform.

Solution 2. 10 mg DSPE-PEG(2000)Carboxylic Acid(1,2-Distearoyl-sn-Glycero-3-Phosphoethanolamine-N-[Carboxy(Polyethylene Glycol)2000] (Ammonium Salt)) and 60 mg mPEG2000PE ([1,2-diacyl-sn-glycero-3-phosphoethanolamine-n-methoxy(polyethylene glycol)-2000] (Ammonium Salt)) lipids were dissolved in 3 ml chloroform solution. It has been found that a ⅙ ratio of carboxy lipid to ammonium salt lipid allows for an optimal number of functional groups, approximately three. However, these ratios may be varied depending on the number of functional groups desired on the semiconductor nanocrystal complex.

Solution 1 and solution 2 were mixed together in a 20 ml vial and the resultant solution dried under N₂. The vial was rotated slowly to make a thin film on the wall while the solution was drying. The vial was heated at 75° C. in a water bath for 2 minutes. To the heated vial, 5 ml deionized water, which has been preheated to 75° C., was added. Then the vial was capped, and the solution was vortexed until all the nanocrystals were dissolved. Then the solution was sonicated for 1 minute.

The solution was transferred into a 15 ml centrifuge tube, centrifuged at 4000 rpm for 5 minutes. The clear supernatant was loaded into a 10 ml syringe, and was filtrated through a 0.2 cm filter. Afterwards, the filtrated solution was loaded into two 11 ml ultracentrifuge tubes and centrifuged at 65000 rpm for 1 hour. The supernatant was removed carefully, and the precipitates re-dissolved into deionized water. The ultracentrifuge purification step was repeated one more time. The pellets were reconstituted into 4 ml deionized water and stored at 4° C.

### EXAMPLE 2c

### Amine Terminated Semiconductor Nanocrystal Complexes

Solution 1. 3 mg (1 mg/ml) CdSe/ZnS core-shell nanocrystals toluene solution (emission at ˜520 nm) was loaded into a 15 ml centrifuge tube, and 10 ml methanol was added. The solution was mixed, and centrifuged at 400 rpm for 3 minutes. The supernatant was removed carefully; and 3 ml of hexane was added to re-dissolve the pellet of the nanocrystals. Then 10 ml methanol was added in the tube to precipitate down the nanocrystals again. The hexane and methanol purification steps were repeated one more time. The precipitate pellet was dried and then re-dissolved in 1 ml chloroform.

Solution 2. 10 mg DSPE-PEG(2000)Amine (1,2-Distearoyl-sn-Glycero-3-Phosphoethanolamine-N-[Amino(Polyethylene Glycol)2000] (Ammonium Salt)) and 60 mg mPEG2000PE ([1,2-diacyl-sn-glycero-3-phosphoethanolamine-n-methoxy(polyethylene glycol)-2000] (Ammonium Salt)) lipids were dissolved in 3 ml chloroform solution. It has been found that a ⅙ ratio of amine lipid to ammonium salt lipid allows for an optimal number of functional groups. However, these ratios may be varied depending on the number of functional groups desired on the semiconductor nanocrystal complex. Solution 1 and solution 2 were mixed together in a 20 ml vial and the resultant solution was dried under N₂. The vial was rotated slowly to make a thin film on the wall while the solution is drying. The vial was heated at 75° C. in a water bath for 2 minutes. To the heated vial, 5 ml deionized water, which had been preheated to 75° C., was added. Then the vial was capped, and the solution was vortexed until all the nanocrystals were dissolved. Then the solution was sonicated for 1 minute.

The solution was transferred into a 15 ml centrifuge tube, centrifuged at 4000 rpm for 5 minutes. The clear supernatant was loaded into a 10 ml syringe, and was filtrated through a 0.2 μm filter. Afterwards, the filtrated solution was loaded into two 11 ml ultracentrifuge tubes, centrifuged at 65000 rpm for 1 hour. The supernatant was removed carefully, and re-dissolved into deionized water. The ultracentrifuge purification step was repeated one more time and the pellets reconstituted into 4 ml deionized water and stored at 4° C.

### EXAMPLE 3

### Semiconductor Nanocrystal Complexes as FRET Donors for the Detection of Molecular Interactions

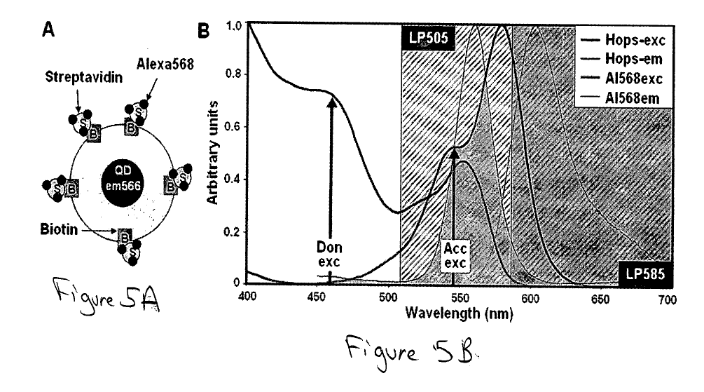

Semiconductor nanocrystal complexes of the present invention were prepared using semiconductor nanocrystals that emit at 566 nm labeled with biotin molecules as the donor molecule and Alexa568-streptavidin (A1568-s) as the acceptor molecule (FIG. 5A). These two molecules show a strong spectral overlap with no acceptor SBT and reduced donor SBT.

FIG. 5(A) depicts a semiconductor nanocrystal complex (emission 566 nm) bound to an A1568-s. FIG. 5(B) depicts normalized excitation and emission spectra of semiconductor complex and Alexa568. Longpass filters (LP505-green- and LP585-red-) were used for collecting donor and acceptor channel emission, respectively, for the purpose of FIG. 5B. In FIG. 5(B) the reference to Hops refers to a semiconductor nanocrystal complex that emits light upon excitation at 566 nm.

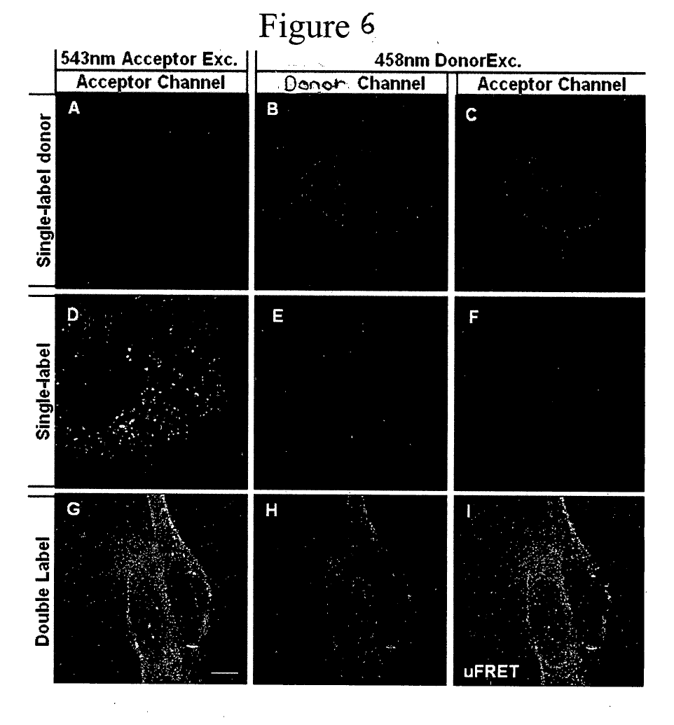

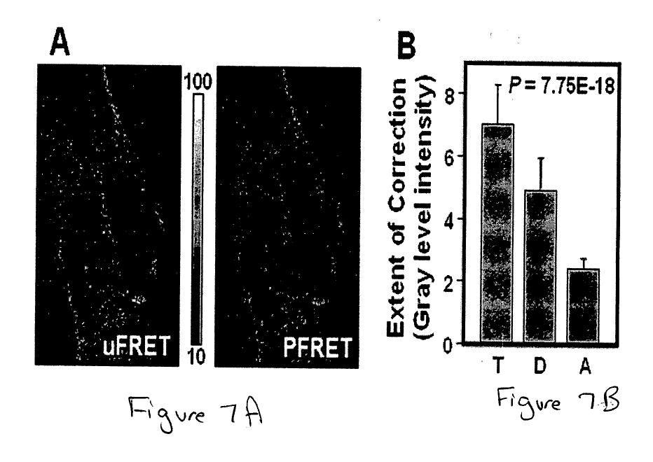

One of the major advantages of using the semiconductor nanocrystal complexes of the present invention as FRET donors is the ability to select donor-acceptor pairs that show strong overlap with reduced or even non-significant SBT. The HY-b/A1568-s pair meets this objective when using the 458 nm laser line as the donor excitation and filters that collect acceptor channel emission beyond ˜600 nm. Here, a 585LP emission filter, available in the Zeiss510 Meta confocal microscope may be used. HY-b/A1568-s complexes were incubated at high concentration with non-polarized MDCK cells for 2 hours at 37° C. As shown in FIG. 6D, some of these complexes were internalized by fluid-phase endocytosis as suggested by the labeled punctuate structures that are analogous to endocytic structures. Plasma membrane (PM) labeling detected in FIG. 6G, indicates non-specific binding or the initial steps of fluid-phase endocytosis.

The nine images shown in FIG. 6 were collected from donor and acceptor single-labeled and double labeled cells using a Zeiss 510META confocal microscope with the following multi-tracking imaging conditions: 8-bit, 512×512 resolution, pinhole ˜143 μm, 458 nm donor and 543 nm acceptor excitation, acceptor emission LP585 and donor emission LP505 filters (FIG. 5B); gain and black levels were kept constant during imaging. Seven of those images (FIGS. 6B-D&F-I) were then processed by a PFRET algorithm to generate the corrected PFRET image (FIG. 7B) that contains the actual energy transfer levels and to calculate E %.

Previously, it has been demonstrated that it is necessary to implement SBT correction methods to generate correctly processed FRET results when using organic fluorophores as acceptor and donor molecules. As discussed above, by selecting a particular semiconductor nanocrystal complex as a donor and an organic fluorophore as an acceptor as well as specific emission filters, the SBT can be significantly reduced while preserving the strong spectral overlap necessary for FRET (FIG. 5B).

The particular FRET pair of (FIG. 5A) was subjected to FRET confocal imaging (FIG. 6) and processing by the PFRET algorithm to measure the extent of SBT correction (i.e. the difference between uFRET and PFRET pixel intensity) and calculated the actual energy transfer and E % levels (FIG. 7). The SBT due to the donor excitation of acceptor fluorophore (lane A) and donor emission bleedthrough into the acceptor channel (lane D) is discriminated in FIG. 7B. As expected, the extent of correction due to both donor and acceptor SBT (lane T) is higher than that due to donor (lane D) or acceptor SBT (lane A) and more importantly the majority of the correction (˜70%) is generated by the donor emission bleedtrough into the acceptor channel (lane D). This SBT can be significantly reduced by using an acceptor emission LP600 filter. This PFRET algorithm may be upgraded to include images FIGS. 6A and 6E to address the potential excitation of donor molecules by the acceptor excitation (FIG. 5B).

FIG. 7A shows the extent of SBT correction. uFRET represents the donor excitation/acceptor channel gray-scale image. PFRET gray-scale image represents the uFRET image after processing by the PFRET custom algorithm (CircusSoft), which removes donor and acceptor SBT. In FIG. 7B T represents the total; D represents the donor; and A represents the acceptor extent of SBT correction (difference between uFRET and PFRET pixel intensity). P-values were obtained using Anova single-factor analysis.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Further, it is appreciated that although a number of problems and deficiencies may be identified above, each embodiment of the present invention may not solve each problem identified in the art. Additionally, to the extent a problem identified in the art or an advantage of the present invention is not cured, solved or lessened by the claimed invention, the solution to such problems or the advantage identified above should not be read into the claimed invention. Furthermore, all references cited herein are incorporated by reference in their entirety.

What is claimed is:

1. A semiconductor nanocrystal complex comprising:
a semiconductor nanocrystal;
a surfactant layer surrounding the semiconductor nanocrystal, the surfactant having a moiety with an affinity for the semiconductor nanocrystal and a moiety with an affinity for a hydrophobic solvent; and
an additional layer having a hydrophobic end for interacting with the surfactant layer and a hydrophilic end, wherein the semiconductor nanocrystal complex is adapted to act as a fluorescence resonance energy transfer (FRET) donor.

2. The semiconductor nanocrystal complex of claim 1, wherein the semiconductor nanocrystal comprises a semiconductor nanocrystal shell overcoating a semiconductor nanocrystal core.

3. The semiconductor nanocrystal complex of claim 1, wherein the hydrophilic end of the additional layer comprises a functional group for coupling to one or more tertiary molecules.

4. The semiconductor nanocrystal complex of claim 3, further comprising a tertiary molecule coupled to the functional group.

5. The semiconductor nanocrystal complex of claim 4, wherein the tertiary molecule is a member of a specific binding pair.

6. The semiconductor nanocrystal complex of claim 5, wherein the member of the specific binding pair is selected from the group consisting of antibody, antigen, hapten, antihapten, biotin, avidin, streptavidin, IgG, protein A, protein G, drug receptor, drug, toxin receptor, toxin, carbohydrate, lectin, peptide receptor, peptide, protein receptor, protein, carbohydrate receptor, carbohydrate, polynucleotide binding protein, polynucleotide, DNA, RNA, aDNA, aRNA, enzyme, substrate.

7. The semiconductor nanocrystal complex of claim 4, wherein the tertiary molecule is selected from the group consisting of an polypeptide, glycopeptide, peptide nucleic acid, oligonucleotide, aptamer, cellular receptor molecule, enzyme cofactor, oligosaccharide, a liposaccharide, a glycolipid, a polymer, a metallic surface, a metallic particle, and a organic dye molecule.

8. The semiconductor nanocrystal complex of claim 1, wherein the distance between the semiconductor nanocrystal and the additional layer is less than 100 Angstroms.

9. The semiconductor nanocrystal complex of claim 8, wherein the distance between the semiconductor nanocrystal and the additional layer is less than 90 Angstroms.

10. The semiconductor nanocrystal complex of claim 9, wherein the distance between the semiconductor nanocrystal and the additional layer is less than 80 Angstroms.

11. The semiconductor nanocrystal complex of claim 10, wherein the distance between the semiconductor nanocrystal and the additional layer is less than 70 Angstroms.

12. The semiconductor nanocrystal complex of claim 3, wherein the distance between the semiconductor nanocrystal and the functional group is less than 100 Angstroms.

13. The semiconductor nanocrystal complex of claim 12, wherein the distance between the semiconductor nanocrystal and the functional group is less than 90 Angstroms.

14. The semiconductor nanocrystal complex of claim 13, wherein the distance between the semiconductor nanocrystal and the functional group is less than 80 Angstroms.

15. The semiconductor nanocrystal complex of claim 14, wherein the distance between the semiconductor nanocrystal and the functional group is less than 70 Angstroms.

16. The semiconductor nanocrystal complex of claim 4, wherein the distance between the semiconductor nanocrystal and the tertiary molecule is less than 100 Angstroms.

17. The semiconductor nanocrystal complex of claim 16, wherein the distance between the semiconductor nanocrystal and the tertiary molecule is less than 90 Angstroms.

18. The semiconductor nanocrystal complex of claim 17, wherein the distance between the semiconductor nanocrystal and the tertiary molecule is less than 80 Angstroms.

19. The semiconductor nanocrystal complex of claim 18, wherein the distance between the semiconductor nanocrystal and the tertiary molecule is less than 70 Angstroms.

20. The semiconductor nanocrystal complex of claim 1, wherein the semiconductor nanocrystal complex has a quantum yield of over 10% as measured under ambient conditions.

21. The semiconductor nanocrystal complex of claim 1, wherein the semiconductor nanocrystal complex has a quantum yield of over 20% as measured under ambient conditions.

22. The semiconductor nanocrystal complex of claim 1, wherein the semiconductor nanocrystal complex has a quantum yield of over 35% as measured under ambient conditions.

23. The semiconductor nanocrystal complex of claim 1, wherein the semiconductor nanocrystal complex has a quantum yield of over 50% as measured under ambient conditions.

24. The semiconductor nanocrystal complex of claim 1, wherein the semiconductor nanocrystal complex has an energy transfer greater than 30%.

25. The semiconductor nanocrystal complex of claim 1, wherein the semiconductor nanocrystal complex has an energy transfer greater than 40%.

26. The semiconductor nanocrystal complex of claim 1, wherein the semiconductor nanocrystal complex has an energy transfer greater than 50%.

27. A method of detecting an acceptor molecule in a aqueous solution comprising:
introducing the semiconductor nanocrystal complex of claim 4 into an aqueous solution;
exciting the semiconductor nanocrystal complex;
determining a light emission from the aqueous solution containing the semiconductor nanocrystal complex; and
detecting the presence of an acceptor molecule in the aqueous solution based on the light emission.

28. The method of claim 27, wherein the distance between the semiconductor nanocrystal complex and the acceptor molecule is between 1 and 10 nanometers.

29. The method of claim 27, wherein the distance between the semiconductor nanocrystal complex and the tertiary molecule is less than 100 Angstroms.

30. The method of claim 27, wherein the semiconductor nanocrystal complex has a quantum yield of over 10%.

31. The method of claim 27, wherein the semiconductor nanocrystal complex has an energy transfer greater than 30%.
